(19) United States
(12) Patent Application Publication
(10) Pub. No.: US 2022/0000913 A1
(43) Pub. Date: Jan. 6, 2022

(54) Enteric Use of Potassium Permanganate in Humans and Animals

(71) Applicants: Sidney Fierro, Midland, TX (US); Frank Fierro, Midland, TX (US)

(72) Inventors: Sidney Fierro, Midland, TX (US); Frank Fierro, Midland, TX (US)

(21) Appl. No.: 16/919,268

(22) Filed: Jul. 2, 2020

(51) Int. Cl. A61K 33/32; A61K 9/08; A61K 9/00; A61K 9/20

(52) U.S. Cl. CPC A61K 33/32; A61K 9/08; A61K 9/20; A61K 9/0078; A61K 9/0053; A61K 9/0019

(57) ABSTRACT

The invention relates generally to the enteric use of potassium permanganate in limited concentrations to treat viral and bacterial infections in humans and animals. More specifically, the invention relates to the enteric use of solutions of potassium permanganate instilled into the body by means of an orally administered liquid or intravascularly administered liquid or by means of a soft mist inhaler or by means of tablets or capsules taken with liquids. Potassium permanganate is a known powerful antiseptic used externally and in the treatment of wounds, etc. What is novel is that potassium permanganate taken internally treats a variety of viral and bacteriological infective agents including SARS-CoV-2.

### CROSS-REFERENCE TO RELATED APPLICATIONS

Not applicable.

### FIELD OF THE INVENTION

The invention relates generally to the enteric use of potassium permanganate in limited concentrations to treat viral and bacterial infections in humans and animals. More specifically, the invention relates to the enteric use of solutions of potassium permanganate instilled into the body by means of an orally administered liquid, an intravascularly administered liquid, by means of a soft mist inhaler, or by means of time release tablets taken with liquids. Potassium permanganate is a known powerful antiseptic used externally and in the treatment of wounds, etc. What is novel is that potassium permanganate taken internally in minute concentrations treats a variety of viral and bacteriological infective agents including SARS-CoV-2.

### BACKGROUND OF THE INVENTION

Potassium permanganate (KMnO₄)(permanganate of potash) is a well-known, effective sanitizing agent that is used externally to disinfect surfaces and debride wounds. It is also used to treat various fungal infections of the foot, impetigo, pemphigus, superficial wounds, dermatitis, and tropical ulcers. Potassium permanganate is a highly corrosive, water-soluble oxidizing antiseptic for cleansing and deodorizing suppurative eczematous reactions and wounds, used in baths and wet bandages. It has been used as an abortifacient, as a urethral irrigation fluid for treatment of gonorrhea, as a fluid for stomach washout in cases of alkaloid poisoning, and in the solid form as a local remedy for snake bite. Formulations include ready-to-use solutions, pellets, tablets, crystals, and powder. Potassium permanganate is one of the World Health Organization's (WHO) Essential Medicines.

But potassium permanganate is well known to be poisonous if administered enterically. For example, in 1910 it was established that 5.0 cc of a 5% solution of potassium permanganate in aqueous or saline solution injected intravenously into dogs caused death in slightly over a minute. Further experiments were conducted where 40.0 cc of a 0.5% solution was injected intravenously, and the 15-pound dog died seven days later exhibiting liver damage, lung congestion, and congested kidneys. Another 15-pound dog died on the second day after 50.0 cc of a 0.5% solution was injected intravenously and was shown on post-mortem to have congested lungs, liver damage, and enlarged kidneys.

Potassium permanganate has an oral LD50 (lethal dose half of the population) of 1,090 mg/kg in rats. Potassium permanganate has an oral LDLo (lethal dose low) of 143 mg/kg in humans. The probable lethal dose in humans is 10.0 g (or about 1.5 teaspoons) of crystals and death usually results from pharyngeal edema and cardiovascular collapse. The lowest recorded instance of an oral human poisoning was 100 mg/kg.

Patients have been admitted to the hospital after ingesting low quantities of potassium permanganate. For example, a case was reported in which 2.5 g of potassium permanganate was ingested and a non-morbid stay of seven days in the hospital occurred. The mass of the patient is not noted in the report but this likely represented no more than a 550 mg/kg event.

Potassium permanganate has been administered topically to treat a variety of conditions including wound cleansing and debridement, nasal wash, gonococcus, vaginal and uterine irrigation, cystitis, etc. Potassium permanganate has been administered enterically to treat a variety of conditions including varicella.

What is needed therefore is an administration method and dosage low enough to be easily tolerated by humans and animals.

What is needed is an administration dosage and method that results in a high enough concentration to be effective to treat SARS-CoV-2.

What is needed is an administration dosage and method that results in a high enough concentration to be effective to treat the common human coronaviruses (229E, NL63, OC43, and HKU1).

What is needed is an administration dosage and method that results in a high enough concentration to be effective to treat the common human seasonal influenza viruses (H1N1, H3N2, Victoria, and Yamagata).

What is needed is an administration dosage and method that results in a high enough concentration to be effective to treat West Nile virus.

### SUMMARY OF THE INVENTION

According to a first embodiment of the present invention, potassium permanganate is orally administered to humans in a low 10 mg/kg per day regimen for up to three days. Also, according to a second embodiment of the present invention, potassium permanganate is orally administered to humans in a very low 5 mg/kg per day regimen for up to three days. Also, according to a third embodiment of the present invention, potassium permanganate is orally administered to humans in an extremely low 1 mg/kg per day regimen for up to three days. The dosages may be administered in aqueous or normal saline solution. Alternately, time release tablets of potassium permanganate crystals may be administered.

Alternately, according to a fourth embodiment of the present invention, potassium permanganate solution is administered intravenously to humans in 10.0 cc per 45.4 kg (100 lbs. body mass) of a low 1:10,000 dilution per day regimen for up to three days. Also, according to a fifth embodiment of the present invention, potassium permanganate is administered intravenously to humans in 10.0 cc per 45.4 kg (100 lbs. body mass) of a very low 1:50,000 dilution per day regimen for up to three days. Also, according to a sixth embodiment of the present invention, potassium permanganate is administered intravenously to humans in 10.0 cc per 45.4 kg (100 lbs. body mass) in an extremely low 1:100,000 dilution per day regimen for up to three days. The dosages may be administered in aqueous or normal saline solution.

Alternately, according to a seventh embodiment of the present invention, potassium permanganate solution is administered by means of a nebulizer to humans in 3.0 ml of a low 1:10,000 dilution per day for up to three days. Also, according to an eighth embodiment of the present invention, potassium permanganate is administered by means of a nebulizer to humans in 3.0 ml of a very low 1:50,000 dilution per day for up to three days. Also, according to a ninth embodiment of the present invention, potassium permanganate is administered by means of a nebulizer to humans in 3.0 ml of an extremely low 1:100,000 dilution per day for up to three days. The inhalant may be administered in aqueous or normal saline solution.

### DETAILED DESCRIPTION OF THE EMBODIMENTS

According to the first embodiment of the present invention, potassium permanganate is orally administered to humans in a low 10 mg/kg per day regimen for three days. Such a regimen has been shown to be effective in treating SARS-CoV-2, infections resulting from the common human coronaviruses (229E, NL63, OC43, and HKU1), infections resulting from the common human seasonal influenza viruses (H1N1, H3N2, Victoria, and Yamagata), and West Nile virus.

Accordingly, a 100 lb. human (45.4 kg) is administered no more than 10 mg/kg (454.0 mg) of potassium permanganate per day orally. The potassium permanganate may be prepared in aqueous or normal saline solution. Alternately, a time release tablet containing of 454.0 mg of potassium permanganate crystals per day may be administered. Ordinarily, administration is timed early in the infection cycle for best results.

According to a second embodiment of the present invention, potassium permanganate is orally administered to humans in a very low 5 mg/kg per day regimen for three days. As before, such a regimen has also been shown to be effective in treating SARS-CoV-2, infections resulting from the common human coronaviruses (229E, NL63, OC43, and HKU1), infections resulting from the common human seasonal influenza viruses (H1N1, H3N2, Victoria, and Yamagata), and West Nile virus.

Accordingly, a 100 lb. human (45.4 kg) is administered no more than 5 mg/kg (227 mg) of potassium permanganate per day orally. The potassium permanganate may be prepared in aqueous or normal saline solution. Alternately, a time release tablet containing 227 mg of potassium permanganate crystals per day may be administered. Ordinarily, administration is timed early in the infection cycle for best results.

According to a third embodiment of the present invention, potassium permanganate is orally administered to humans in an extremely low 1 mg/kg per day regimen for three days. As above, such a regimen has also been shown to be effective in treating SARS-CoV-2, infections resulting from the common human coronaviruses (229E, NL63, OC43, and HKU1), infections resulting from the common human seasonal influenza viruses (H1N1, H3N2, Victoria, and Yamagata), and West Nile virus.

Accordingly, a 100 lb. human (45.4 kg) is administered no more than 1 mg/kg (45.4 mg) of potassium permanganate per day orally. The potassium permanganate may be prepared in aqueous or normal saline solution. Alternately, a capsule or a time release tablet containing of 45.4 mg of potassium permanganate crystals per day may be administered. Ordinarily, administration is timed early in the infection cycle for best results.

According to the fourth embodiment of the present invention, potassium permanganate is intravenously administered to humans in 10.0 cc per 45.4 kg (100 lbs. body mass) of a low 1:10,000 dilution per day regimen for three days. Such a regimen has been shown to be effective in treating SARS-CoV-2, infections resulting from the common human coronaviruses (229E, NL63, OC43, and HKU1), infections resulting from the common human seasonal influenza viruses (H1N1, H3N2, Victoria, and Yamagata), and West Nile virus.

Accordingly, a 100 lb. human (45.4 kg) is administered no more than 10.0 cc per 45.4 kg (100 lbs. body mass) of a low 1:10,000 dilution of potassium permanganate per day intravenously. The potassium permanganate may be prepared in aqueous or normal saline solution. Ordinarily, administration is timed early in the infection cycle for best results.

According to a fifth embodiment of the present invention, potassium permanganate is intravenously administered to humans in 10.0 cc per 45.4 kg (100 lbs. body mass) of a very low 1:50,000 dilution per day for three days. As before, such a regimen has also been shown to be effective in treating SARS-CoV-2, infections resulting from the common human coronaviruses (229E, NL63, OC43, and HKU1), infections resulting from the common human seasonal influenza viruses (H1N1, H3N2, Victoria, and Yamagata), and West Nile virus.

Accordingly, a 100 lb. human (45.4 kg) is administered no more than 10.0 cc per 45.4 kg (100 lbs. body mass) of a very low 1:50,000 dilution of potassium permanganate per day intravenously. The potassium permanganate may be prepared in aqueous or normal saline solution. Ordinarily, administration is timed early in the infection cycle for best results.

According to a sixth embodiment of the present invention, potassium permanganate is intravenously administered to humans in 10.0 cc per 45.4 kg (100 lbs. body mass) of an extremely low 1:100,000 dilution per day for three days. As above, such a regimen has also been shown to be effective in treating SARS-CoV-2, infections resulting from the common human coronaviruses (229E, NL63, OC43, and HKU1), infections resulting from the common human seasonal influenza viruses (H1N1, H3N2, Victoria, and Yamagata), and West Nile virus.

Accordingly, a 100 lb. human (45.4 kg) is administered no more than 10.0 cc per 45.4 kg (100 lbs. body mass) of an extremely low 1:100,000 dilution per day intravenously. The potassium permanganate may be prepared in aqueous or normal saline solution. Ordinarily, administration is timed early in the infection cycle for best results.

According to the seventh embodiment of the present invention, potassium permanganate is administered by inhalation by means of a cold-mist inhaler or a nebulizer to adult humans in 3.0 ml of a low 1:10,000 dilution per day regimen for three days. Such a regimen has been shown to be effective in treating SARS-CoV-2, infections resulting from the common human coronaviruses (229E, NL63, OC43, and HKU1), infections resulting from the common human seasonal influenza viruses (H1N1, H3N2, Victoria, and Yamagata), and West Nile virus.

Accordingly, an adult human is administered no more than 3.0 ml of a low 1:10,000 dilution of potassium permanganate per day by means of a cold-mist inhaler or a nebulizer. The potassium permanganate may be prepared in aqueous or normal saline solution. Ordinarily, administration is timed early in the infection cycle for best results.

According to the eighth embodiment of the present invention, potassium permanganate is administered by inhalation by means of a cold-mist inhaler or a nebulizer to adult humans in 3.0 ml of a very low 1:50,000 dilution per day regimen for three days. Such a regimen has been shown to be effective in treating SARS-CoV-2, infections resulting from the common human coronaviruses (229E, NL63, OC43, and HKU1), infections resulting from the common human seasonal influenza viruses (H1N1, H3N2, Victoria, and Yamagata), and West Nile virus.

Accordingly, an adult human is administered no more than 3.0 ml of a very low 1:50,000 dilution of potassium permanganate per day by means of a cold-mist inhaler or a nebulizer. The potassium permanganate may be prepared in aqueous or normal saline solution. Ordinarily, administration is timed early in the infection cycle for best results.

According to the ninth embodiment of the present invention, potassium permanganate is administered by inhalation by means of a cold-mist inhaler or a nebulizer to adult humans in 3.0 ml of an extremely low 1:100,000 dilution per day regimen for three days. Such a regimen has been shown to be effective in treating SARS-CoV-2, infections resulting from the common human coronaviruses (229E, NL63, OC43, and HKU1), infections resulting from the common human seasonal influenza viruses (H1N1, H3N2, Victoria, and Yamagata), and West Nile virus.

Accordingly, an adult human is administered no more than 3.0 ml of an extremely low 1:100,000 dilution of potassium permanganate per day by means of a cold-mist inhaler or a nebulizer. The potassium permanganate may be prepared in aqueous or normal saline solution. Ordinarily, administration is timed early in the infection cycle for best results.

It will be obvious to those having skill in the art that different concentrations within the range between the minimum and the maximum disclosed above may be administered to humans. Also, it will be obvious to those having skill in the art that different concentrations within the range between the minimum and the maximum disclosed may be administered to animals. Also, it will be obvious to those having skill in the art that different concentrations within the range between the minimum and the maximum disclosed may be administered to humans and animals for the treatment of other infective diseases and conditions. Also, it will be obvious to those having skill in the art that different dosages may be calculated based on the age and/or weight of the patient. For example, a 200 lb. human would be dosed at twice the specified 100 lb. rate. Also, a 50-pound human child would be dosed at one-half the specified 100 lb. rate.

1. A method of treating infective diseases of the lung by administering to a patient in need thereof a composition comprising potassium permanganate in an aqueous or normal saline solution orally or by intravenous injection.

2. The method of claim 1, wherein the composition is administered orally by means of an enterically consumed dosage.

3. (canceled)

4. The method of claim 1, wherein the composition is administered by means of an intravenous injection.

5. (canceled)

6. The method of claim 2, wherein the administered dosage of potassium permanganate is less than 10 mg per 1 kg of the body weight of the patient per day.

7. (canceled)

8. The method of claim 4, wherein the administered dosage of potassium permanganate is less than 10 mg per 1 kg of the body weight of the patient per day.

9. (canceled)

10. A method of treating infective diseases of the lung by administering to a patient in need thereof an aqueous or normal saline solution of potassium permanganate, orally or by intravenous injection, wherein the patient is a human.

11. A method of treating infective diseases of the lung by administering to a patient in need thereof an aqueous or normal saline solution of potassium permanganate, orally or by intravenous injection, wherein the patient is an animal.

12. The method of treating infective diseases of the lung of claim 6, wherein the course of treatment lasts 1 day.

13. The method of treating infective diseases of the lung of claim 6, wherein the course of treatment lasts 2 days.

14. The method of treating infective diseases of the lung of claim 6, wherein the course of treatment lasts 3 days.

15.-17. (canceled)

18. The method of treating infective diseases of the lung of claim 8, wherein the course of treatment lasts 1 day.

19. The method of treating infective diseases of the lung of claim 8, wherein the course of treatment lasts 2 days.

20. The method of treating infective diseases of the lung of claim 8, wherein the course of treatment lasts 3 days.
